(19) United States
(12) Patent Application Publication
(10) Pub. No.: US 2025/0182907 A1
(43) Pub. Date: Jun. 5, 2025

(54) ELECTRONIC DEVICE FOR REINFORCEMENT LEARNING RELATED TO MEDICAL DATA AND METHOD FOR OPERATING THE SAME

(71) Applicant: Electronics and Telecommunications Research Institute, Daejeon (KR)

(72) Inventors: Do Hyeun KIM, Goyang-si (KR); Hwin Dol PARK, Daejeon (KR); Jae Hun CHOI, Sejong-si (KR)

(73) Assignee: Electronics and Telecommunications Research Institute, Daejeon (KR)

(21) Appl. No.: 18/960,348

(22) Filed: Nov. 26, 2024

(30) Foreign Application Priority Data
Dec. 1, 2023 (KR) 10-2023-0172451

(51) Int. Cl. G16H 50/50; G16H 10/60

(52) U.S. Cl. CPC G16H 50/50; G16H 10/60

(57) ABSTRACT

An electronic device for reinforcement learning related to medical data and a method of operating the same are provided. The method includes obtaining an actual event related to medical data of a patient and a virtual event generated based on the actual event, determining a probability of the virtual event occurring, based on the medical data, and based on the actual event, the virtual event, and the probability of the virtual event, training a model to determine a state value for a reward of an action that is performed in a current state of the patient.

### CROSS-REFERENCE TO RELATED APPLICATION

This application claims the benefit of Korean Patent Application No. 10-2023-0172451, filed on Dec. 1, 2023, in the Korean Intellectual Property Office, the entire disclosure of which is incorporated herein by reference for all purposes.

### BACKGROUND

### 1. Field of the Invention

One or more embodiments relate to an electronic device for reinforcement learning related to medical data and a method of operating the same.

### 2. Description of the Related Art

Research on reinforcement learning, as a branch of machine learning, has been conducted to determine appropriate actions for a given environment. A structure of the reinforcement learning may include an environment and an agent that performs actions. The agent may determine an action in a given environment and as the environment changes according to the determined action, may obtain a reward according to the changed environment. The reinforcement learning may aim to learn a policy that determines an optimal action for a given state by maximizing the reward. In other words, the agent of the reinforcement learning may obtain the reward according to the action through interaction with the environment, and a model of the reinforcement learning may learn a policy that maximizes the reward by learning a series of experiences.

### SUMMARY

In the medical field, determining an appropriate medical action for a current state of a patient may be difficult and influenced by differences in experience and knowledge of professionals. In addition, the amount of medical data obtained for the patient may be limited, so determining an appropriate medical action may require significant time and effort.

According to various embodiments, a virtual event may be generated based on an actual event related to medical data of a patient, and a probability that the virtual event occurs may be determined.

According to various embodiments, a model may be trained to determine a state value for a reward of an action that is performed in the current state of the patient, based on the actual event, the virtual event, and the probability of the virtual event.

According to various embodiments, the state value for the reward of the action that is performed in the current state of the patient may be determined through a model trained through reinforcement learning.

According to various embodiments, an action that is appropriate to the current state of the patient may be determined based on a predicted state value for the reward.

Other objects and advantages of the present disclosure can be understood by the following description and will become more apparent by the embodiments of the present disclosure. In addition, it will be apparent that the objects and advantages of the present disclosure can be readily realized by the means and combinations thereof recited in the claims.

According to an aspect, there is provided a method of operating an electronic device, the method including obtaining an actual event related to medical data of a patient and a virtual event generated based on the actual event, determining a probability of the virtual event occurring, based on the medical data, and based on the actual event, the virtual event, and the probability of the virtual event, training a model to determine a state value for a reward of an action that is performed in a current state of the patient.

The training of the model may include, in the case of the actual event, determining a predicted value of the reward as the state value and in the case of the virtual event, determining the state value so that the reward is predicted according to the probability of the virtual event.

The training of the model may include training the model to determine the state value by using a Bellman equation, which considers the probability of the virtual event.

The training of the model further may include, based on the state value, training the model to select a policy for determining an action appropriate for the current state.

The determining of the probability may include, based on a database in which the medical data is stored, determining the probability through a model configured to compare the virtual event to distribution of actual events in the database.

The virtual event may be an event in which one or more features are selected from the actual event and an event that is generated by the selected one or more features and is different from the actual event.

The virtual event may include information about a next state of the patient predicted for the action performed in the current state of the patient.

The medical data may include information about the current state of the patient and the action.

According to another aspect, there is provided a method of operating an electronic device, the method including obtaining a current state of a patient and determining a state value for a reward of an action that is performed in the current state of the patient from a model to which an actual event related to medical data of the patient, a virtual event generated based on the actual event, and a probability of the virtual event occurring are input.

The method may further include determining an action appropriate for the current state, based on the state value.

The probability may be determined, based on a database in which the medical data is stored, through a model configured to compare the virtual event to distribution of actual events in the database.

The virtual event may be an event in which one or more features are selected from the actual event and an event that is generated by the selected one or more features and is different from the actual event.

According to another aspect, there is provided an electronic device including a processor configured to obtain an actual event related to medical data of a patient and a virtual event generated based on the actual event, determine a probability of the virtual event occurring, based on the medical data, and based on the actual event, the virtual event, and the probability of the virtual event, train a model to determine a state value for a reward of a medical action that is performed in a current state of the patient.

The processor may be configured to, in the case of the actual event, determine a predicted value of the reward as the state value and in the case of the virtual event, determine the state value so that the reward is predicted according to the probability of the virtual event.

The processor may be configured to train the model to determine the state value by using a Bellman equation, which considers the probability of the virtual event.

The processor may be configured to, based on the state value, train the model to select a policy for determining a medical action appropriate for the current state.

The processor may be configured to, based on a database in which the medical data is stored, determine the probability through a model configured to compare the virtual event to distribution of actual events in the database.

The virtual event may be an event in which one or more features are selected from the actual event and an event that is generated by the selected one or more features and is different from the actual event.

The virtual event may include information about a next state of the patient predicted for the action performed in the current state of the patient.

The medical data may include information about the current state of the patient and the action.

Additional aspects of embodiments will be set forth in part in the description which follows and, in part, will be apparent from the description, or may be learned by practice of the disclosure.

According to various embodiments, an action that is appropriate to a current state of a patient may be determined by training a model through reinforcement learning based on medical data.

According to various embodiments, issues caused by limited medical data may be solved and performance may be improved, by obtaining and utilizing a virtual event based on an actual event.

According to various embodiments, by considering a probability of a virtual event, data unrelated to an actual event may be excluded in reinforcement learning, reward overestimation may be prevented, and an actual treatment policy of a doctor may be referenced.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a diagram illustrating an interaction between an environment and an agent, according to an embodiment;

FIG. 2 is a diagram illustrating a schematic structure of an electronic device, according to an embodiment;

FIG. 3 is a diagram illustrating a flow in which an electronic device trains a model and determines an action through the trained model, according to an embodiment;

FIG. 4 is a diagram illustrating an overall structure of an electronic device, according to an embodiment;

FIG. 5 is a diagram illustrating an operation of a virtual event module of an electronic device, according to an embodiment;

FIG. 6 is a diagram illustrating an operation of generating a virtual event by an electronic device, according to an embodiment;

FIG. 7 is a diagram illustrating an operation of a reinforcement learning module of an electronic device, according to an embodiment;

FIG. 8 is a diagram illustrating an operation of obtaining a virtual event through a state prediction module by an electronic device, according to an embodiment;

FIG. 9 is a schematic flowchart of a training operation of an electronic device for reinforcement learning, according to an embodiment;

FIG. 10 is a schematic flowchart of an inference operation of an electronic device for reinforcement learning, according to an embodiment; and

FIG. 11 is a schematic block diagram of an electronic device for reinforcement learning, according to an embodiment.

### DETAILED DESCRIPTION

The following detailed structural or functional description is provided as an example only and various alterations and modifications may be made to the embodiments. Accordingly, the embodiments are not construed as limited to the disclosure and should be understood to include all changes, equivalents, and replacements within the idea and the technical scope of the disclosure.

As used herein, “A or B”, “at least one of A and B”, “at least one of A or B”, “A, B or C”, “at least one of A, B and C”, “at least one of A, B, or C”, and “one or a combination of at least two of A, B, and C,” each of which may include any one of the items listed together in the corresponding one of the phrases, or all possible combinations thereof. Although terms, such as first, second, and the like are used to describe various components, the components are not limited to the terms. These terms should be used only to distinguish one component from another component. For example, a first component may be referred to as a second component, and similarly the second component may also be referred to as the first component.

It should be noted that if one component is described as being “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which the present disclosure pertains. Terms, such as those defined in commonly used dictionaries, should be construed to have meanings matching with contextual meanings in the relevant art, and are not to be construed to have an ideal or excessively formal meaning unless otherwise defined herein.

Hereinafter, embodiments will be described in detail with reference to the accompanying drawings. When describing the embodiments with reference to the accompanying drawings, like reference numerals refer to like components and a repeated description related thereto will be omitted.

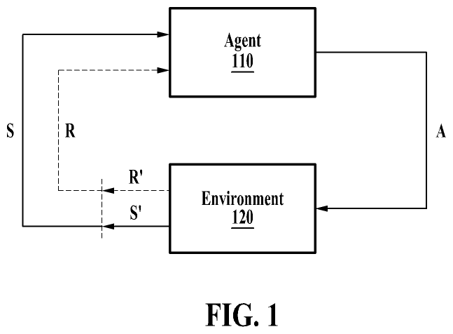

FIG. 1 is a diagram illustrating an interaction between an environment and an agent, according to an embodiment.

Referring to FIG. 1, an example of a structure of an interaction between an environment 120 given and an agent 110 for reinforcement learning is shown. Referring to FIG. 1, S may represent a current state, R may represent a reward for a state transitioning to S, A may represent an action of the agent 110, S′ may represent a next state according to the action, and R′ may represent a reward for the state transitioning from S to S′.

The agent 110 may be a subject that makes decisions in response to the environment 120 given in the reinforcement learning and may include a training algorithm. The agent 110 may interact with the environment 120 and may select an action A to achieve a goal in the current state S.

The environment 120 may be an external system or a situation with which the agent 110 interacts and may provide the next state and the reward, in response to the action of the agent 110. The environment 120 may provide the next state S′ and the reward R′ according to the state transition, in response to the action A of the agent 110.

In the reinforcement learning, an optimal policy may be a policy, among one or more policies, for obtaining maximum rewards when the agent 110 interacts with the environment 120 in the environment 120 given. To calculate a predicted value for the reward in the reinforcement learning, a Bellman equation may be used as shown in Equation 1 below.

$$Q ( s , a ) = {\sum }_{{s}^{′}} {P}_{{ss}^{′}} [ {R}_{{ss}^{′}} + γ {\sum }_{{a}^{′}} π ( {s}^{′} , {a}^{′} ) Q ( {s}^{′} , {a}^{′} ) ] \qquad [ Equation 1 ]$$

Here, s may represent the current state, a may represent the action performed in the current state s, s′ may represent a next state transitioned according to the action a, a′ may represent a next action performed in the next state s′, γ may represent a depreciation rate, R_{ss′} may represent a reward for the state transitioning from s to s′, P_{ss′} may represent a probability of the state transitioning from s to s′, π(s′, a′) may represent a probability of determining the action a′ in the state s′ according to a policy, Q(s, a) may represent a predicted value that may be obtained by performing the action a′ in the state s′, and Q(s′, a′) may represent a predicted value that may be obtained by performing the action a′ in the state s′.

In reinforcement learning, an event may represent each operation in which the agent 110 interacts with the environment 120. In other words, an event may be a single operation of interaction between the agent 110 and the environment 120, which may represent S, A, R, and S′. According to an embodiment, an actual event may be an event related to medical data of a patient and may represent an event about a state of the patient and a medical action that have actually occurred. A virtual event may be an event that is generated based on the actual event and may be an event about a state of the patient or a medical action generated by an electronic device. According to an embodiment, the actual event and the virtual event may be events for an electric medical record (EMR).

In reinforcement learning, an episode may represent a series of state changes and action records of the agent 110 interacting with the environment 120. In other words, the episode may represent a sequence of interactions in which the agent 110 starts from a starting state and reaches a goal or until a certain condition is satisfied. According to an embodiment, the episode may be an episode of a series of medical actions, representing a record of states of the patient and medical actions from the start of initial treatment until the end of treatment. According to an embodiment, the episode may be an episode of the EMR.

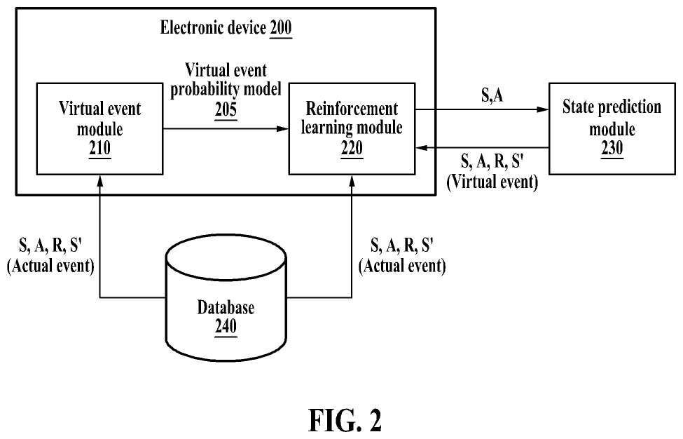

The electronic device may train a model to determine a state value, which is a predicted value of the state of the patient and a reward for the action, based on the actual event or the virtual event. In addition, through the trained model, the electronic device may determine the state value of the reward for the action for the current state of the patient, and an appropriate action may be determined. The specific operations of the electronic device are described in detail below with reference to FIGS. 2 to 11.

FIG. 2 is a diagram illustrating a schematic structure of an electronic device, according to an embodiment.

Referring to FIG. 2, an example of a structure is shown in which an electronic device 200 including a virtual event module 210 and a reinforcement learning module 220 performs reinforcement learning on a model by using a state prediction module 230 and a database 240 storing medical data.

The virtual event module 210 may train the model using the medical data in the database 240. According to an embodiment, the virtual event module 210 may train a model for generating a virtual event, based on an actual event related to the medical data. In addition, the virtual event module 210 may train a model for determining a probability that the generated virtual event occurs. The virtual event module 210 may provide a trained virtual event probability model 205 to the reinforcement learning module 220.

The reinforcement learning module 220 may train the model using the actual event and the virtual event. According to an embodiment, the reinforcement learning module 220 may train the model to determine a state value, which is a predicted value for a reward, based on the actual event, the virtual event, and the probability. In addition, the reinforcement learning module 220 may determine an appropriate action for a current state of a patient through the trained model.

The state prediction module 230 may predict a next state of the patient and the reward by receiving the current state of the patient and an action determined by the model trained through reinforcement learning. In other words, the state prediction module 230 may predict S′ and R using S and A received from the reinforcement learning module 220 and may transmit the virtual event including S, A, S′, and R to the reinforcement learning module 220. An event for the predicted next state and the reward may be classified as a virtual event because the event is not an event that has actually occurred.

The database 240 may store the medical data of the patient and may transmit the actual event related to the medical data to the virtual event module 210 and the reinforcement learning module 220. The actual event transmitted by the database 240 may include S, A, R and S′.

In FIG. 2, the state prediction module 230 and the database 240 are shown as not included in the electronic device 200 but may be included in the electronic device 200 and used for reinforcement learning depending on embodiments.

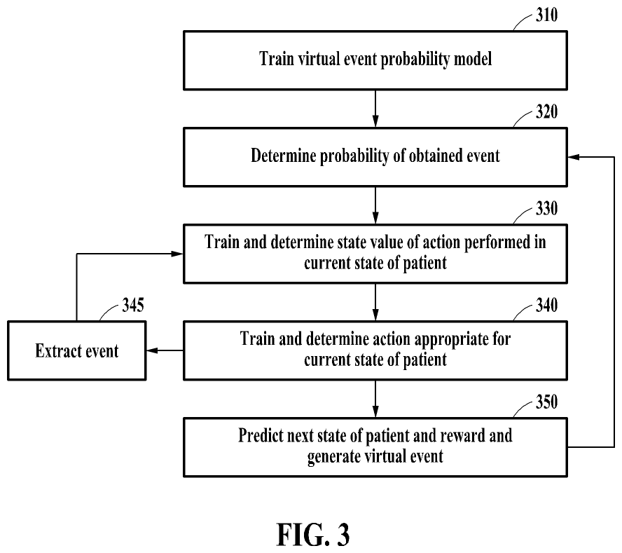

FIG. 3 is a diagram illustrating a flow in which an electronic device trains a model and determines an action through the trained model, according to an embodiment.

In operation 310, an electronic device may train a virtual event probability model that determines a probability that a virtual event occurs.

In operation 320, when an obtained event is the virtual event, the electronic device may determine the probability of the event occurring, through the virtual event probability model. The electronic device may train a model to reflect the probability of the virtual event more on training as the probability of the virtual event increases.

In operation 330, the electronic device may train the model to determine a state value of an action performed in a current state of a patient and may determine the state value. In other words, the electronic device may train the model to determine an expected reward value that may be obtained when a specific treatment is performed in the current state of the patient.

In operation 340, the electronic device may train the model, based on the state value, to determine an action appropriate for the current state of the patient and may determine an action appropriate for a current state of the obtained event.

In operation 345, the electronic device may extract an event about the current state of the patient and the action and may use the event for state value training.

In operation 350, the electronic device may predict a next state of the patient and a reward, based on the determined action, and may generate the virtual event. The next state and the reward may be obtained by a state prediction module. The virtual event may include the current state of the patient, the determined action, the next state, and the reward. The electronic device may train the model by inputting an actual event and the virtual event to the model until the training is complete.

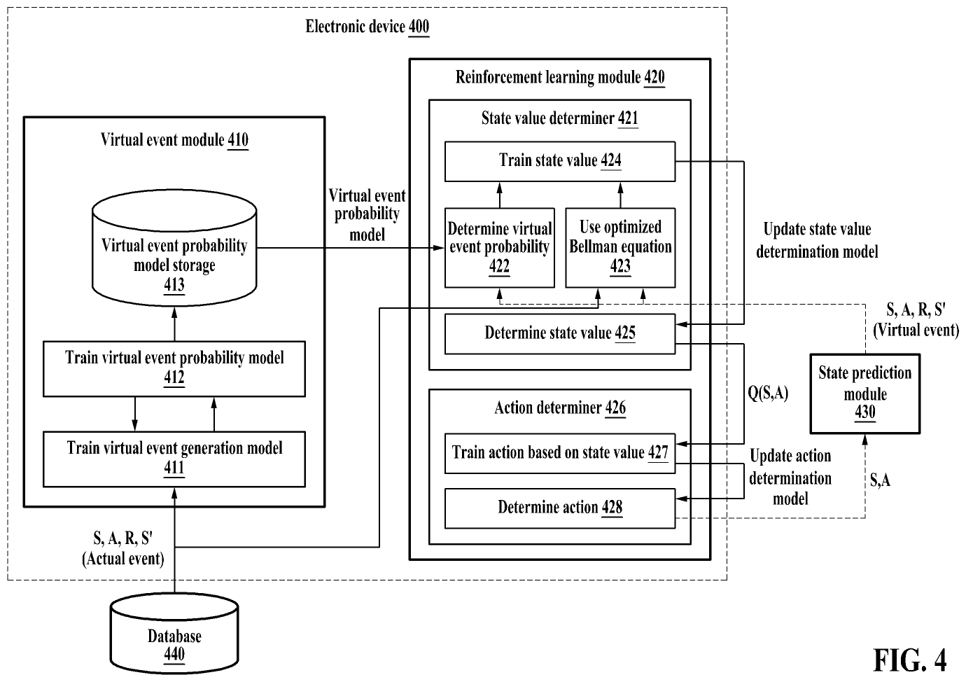

FIG. 4 is a diagram illustrating an overall structure of an electronic device, according to an embodiment.

Referring to FIG. 4, an example of an overall structure and operations of each module is shown in which an electronic device 400 including a virtual event module 410 and a reinforcement learning module 420 performs reinforcement learning on a model by using a state prediction module 430 and a database 440 storing medical data.

In operation 411, the virtual event module 410 of the electronic device 400 may train a virtual event generation model to generate a virtual event, based on an actual event related to the medical data in the database 440. Through the trained model, the state prediction module 430 may determine a next state of a patient and a reward to generate the virtual event.

In operation 412, the virtual event module 410 of the electronic device 400 may train a virtual event probability model to determine a probability of the generated virtual event occurring, based on the medical data. The trained virtual event probability model may be stored in a virtual event probability model storage 413.

The reinforcement learning module 420 may include a state value determiner 421 and an action determiner 426.

The state value determiner 421 may be configured to determine a state value that represents a predicted value for the reward of an action performed in a current state of the patient. In operation 422, the state value determiner 421 may determine the probability of the virtual event received through the virtual event probability model. In operation 423, the state value determiner 421 may use an optimized Bellman equation according to the probability of the received virtual event. In operation 424, the state value determiner 421 may train a state value determination model to predict an expected reward of the action, based on the received actual event, the virtual event, and the probability. In operation 425, the state value determiner 421 may determine, through the state value determination model, the state value by predicting the reward of actions that may be performed in the current state of the patient.

The action determiner 426 may be configured to determine the action based on the determined state value. In operation 427, the action determiner 426 may train an action determination model to determine an action appropriate for the current state of the patient. In operation 428, the action determiner 426 may determine the action appropriate for the current state of the patient through the action determination model.

The state prediction module 430 may predict the next state of the patient and the reward by receiving the current state of the patient and the action determined by the model trained through reinforcement learning. A predicted result may be used as the virtual event by the state value determiner 421.

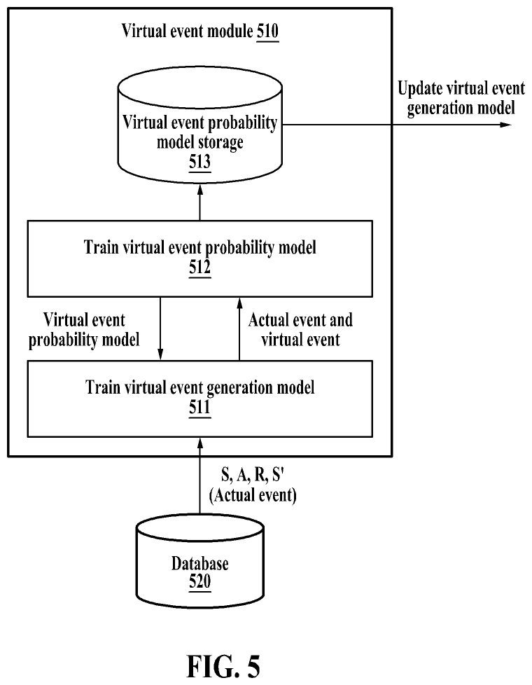

The specific operations of each module are described in detail below with reference to FIGS. 5 to 8.

FIG. 5 is a diagram illustrating an operation of a virtual event module of an electronic device, according to an embodiment.

Referring to FIG. 5, an example of a virtual event module 510 that performs operations 511 and 512 by using medical data in a database 520 is shown.

In operation 511, the virtual event module 510 may train a virtual event generation model to generate a virtual event, based on an actual event related to the medical data in the database 520. The virtual event module 510 may use the actual event and the generated virtual event to train a virtual event probability model. The virtual event generation model may generate the virtual event similar to the actual event by using the actual event as input.

The virtual event generation model may be updated and trained to minimize the difference between the input actual event and the generated virtual event. Alternatively, the virtual event generation model may be trained such that the virtual event generation model is updated in a positive direction when a classification of an event fails in the virtual event probability model and the virtual event generation model is updated in a negative direction when the classification is successful. The virtual event generation model may be trained using, for example, a variational autoencoder-generative adversarial network (VAE-GAN) or an autoencoder-generative adversarial network (AE-GAN).

In operation 512, the virtual event module 510 may train the virtual event probability model to determine a probability of the generated virtual event occurring, based on the medical data in the database 520. The virtual event probability model may be trained using the actual event and the virtual event as input in a method of supervised learning that distinguishes between the two events. In addition, the virtual event module 510 may update the virtual event generation model based on the probability determined for the virtual event. The virtual event probability model generated may be stored in a virtual event probability model storage 513 so that the actual event and the virtual event may be distinguished and the probability of the virtual event may be determined.

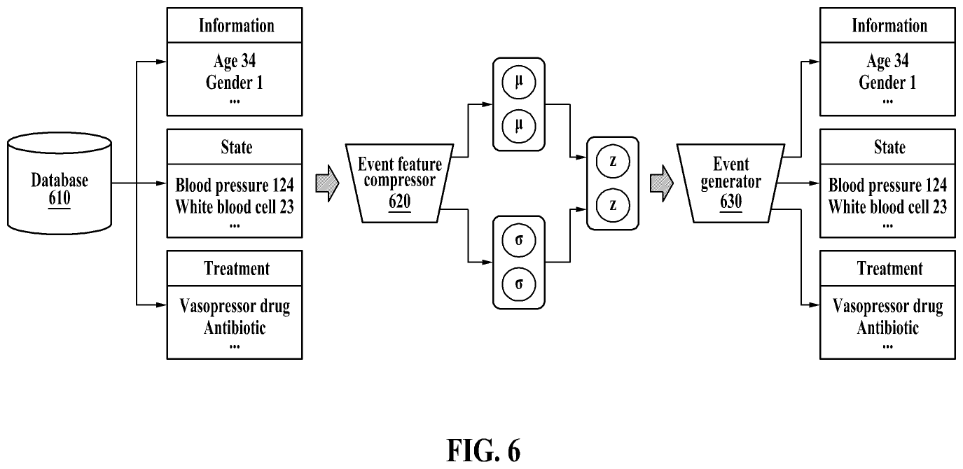

FIG. 6 is a diagram illustrating an operation of generating a virtual event by an electronic device, according to an embodiment.

Referring to FIG. 6, an example of an operation of a virtual event generation model that generates a virtual event by an event feature compressor 620 and an event generator 630 using an actual event in a database 610 is shown.

The database 610 may be a storage for storing medical data and may provide information of the actual event to the event feature compressor 620. For example, the database 610 may provide information about a patient of the actual event, information about a state of the patient, or information about treatment of the patient.

The event feature compressor 620 may use a function that reduces the actual event having N-dimensional data to Z-dimensional data in which Z is less than N. By reducing information of the actual event to Z dimension, the event feature compressor 620 may extract only key information and may prevent a virtual event identical to the input actual event from being generated.

The event generator 630 may receive the data reduced to the Z dimension and may restore the data to the N-dimensional data. The event generator 630 may generate a virtual event that is different from the actual event by using the restored data.

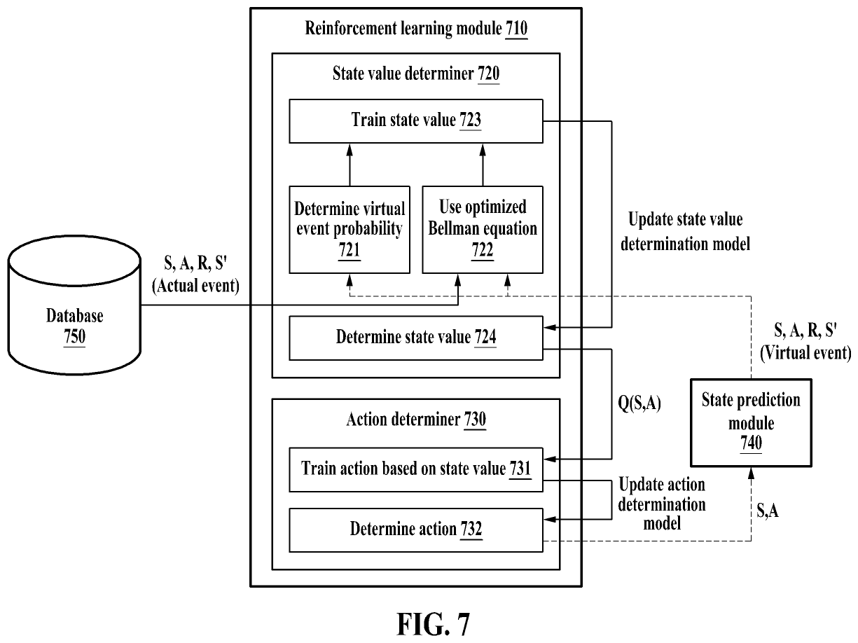

FIG. 7 is a diagram illustrating an operation of a reinforcement learning module of an electronic device, according to an embodiment.

Referring to FIG. 7, a reinforcement learning module 710 including a state value determiner 720 for performing operations 721 to 724 and an action determiner 730 for performing operations 731 and 732, a state prediction module 740, and a database 750 are shown.

The state value determiner 720 may receive an actual event in the database 750 or a virtual event generated from the state prediction module 740 and may predict an expected reward that may be obtained through an action performed in a current state of a patient. In operation 723, the state value determiner 720 may train and update a state value determination model through operations 721 and 722.

When an action policy function π(S, A) is provided and an action a is selected in a current state s, the expected reward may be the sum of rewards that may be sequentially obtained by acting according to the action policy function until an episode ends. Here, the action policy function π(S, A) may represent a probability of selecting an action A in a state S according to an action policy. According to an embodiment, the expected reward may be determined by multiplying a reward that may be obtained later by a depreciation rate, as shown in Equation 2 below.

$${G}_{t} = {R}_{t + 1} + γ {R}_{t + 2} + … = {\sum }_{k = 0}^{∞} {γ}^{k} {R}_{t + k + 1} \qquad [ Equation 2 ]$$

Here, Gₜ may represent the expected reward, γ may represent the depreciation rate, and R may represent a reward for each event.

Since the expected reward may be determined only when the reward is determined by the end of the episode, it may be difficult to determine the expected reward in the current state. A state value determination model may predict the expected reward when the current state and the action are provided. The state value may represent an expected reward value predicted. The larger the expected reward value Q(S, A) predicted for the current state S and the action A, the higher the effect of the action A.

In operation 722, the state value determiner 720 may update the state value determination model by using a general Bellman equation when the actual event is input and using an optimized Bellman equation as shown in Equation 3 below when the virtual event is input.

$${\hat{Q}}^{k + 1} ← arg {min}_{Q} {E}_{s , a , {s}^{′} \sim D , M} [ λ * \vert Q ( s , a ) \vert + ( 1 - λ ) * {( r ( s , a ) + γ {E}_{{a}^{′} \sim {\hat{π}}^{k} ( a ′ \vert s ′ )} [ {\hat{Q}}^{k} ( {s}^{′} , {a}^{′} ) ] - Q ( s , a ) )}^{2} ] \qquad [ Equation 3 ]$$

Here, λ may be a value between 0 and 1 and may represent a probability that is determined through a virtual event probability model, in operation 721. Q(s, a) may represent a predicted value of the expected reward that may be obtained by performing the action a in the state s according to the action policy.

In Equation 3, if λ is 1, the state value determiner 720 may update the state value determination model as shown in Equation 4 below.

$${\hat{Q}}^{k + 1} ← arg {min}_{Q} {E}_{s , a , {s}^{′} \sim D , M} [ λ * \vert Q ( s , a ) \vert ] \qquad [ Equation 4 ]$$

The closer λ is to 1, the higher the probability that the received event is the virtual event, and the state value determination model may predict the expected reward to be minimized. In other words, λ being closer to 1 may indicate that the current state s or the action a is less realistic. Through λ, the state value determination model may prevent a phenomenon of reward overestimation, in which the expected reward for an unknown state or action is overestimated and accordingly, performance of the model is degraded, and may prevent learning of an action policy that is far from an actual treatment policy of a doctor. On the contrary, when λ is 0, the state value determination model may be updated through an equation identical to the general Bellman equation as in the actual event.

The action determiner 730 may train an action determination model to determine a policy for an action appropriate for the current state, based on the determined state value, and may determine an action appropriate for the received event. In operation 731, training of the action determination model may be performed as shown in Equation 5 below.

$$θ = θ + {𝔼}_{π} [ {Q}^{π} ( s , a ) {∇}_{θ} ln {π}_{θ} ( a \vert s ) ] \qquad [ Equation 5 ]$$

Here, θ may represent a parameter learned about a policy π. The action determination model may be updated by adding a value obtained by differentiating the policy π by θ to a value obtained by multiplying Q(s, a) when the action a is selected for the state s given for the parameter θ, as shown in Equation 5. In other words, as the value of Q(s, a) increases, convergence of the policy π may be induced to select the action a.

In operation 732, the action determiner 730 may determine the action appropriate for the current state of the patient, which is received through the updated action determination model.

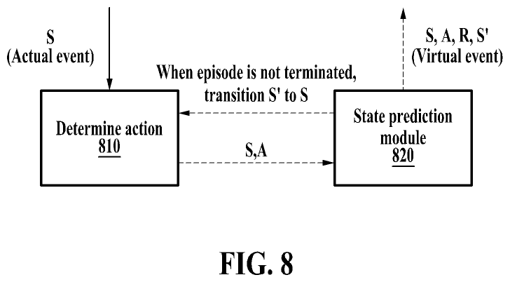

FIG. 8 is a diagram illustrating an operation of obtaining a virtual event through a state prediction module by an electronic device, according to an embodiment.

Referring to FIG. 8, an example of an operation 810 of an action determiner that interacts with a state prediction module 820 for a state S of an actual event is shown.

The action determiner may not only train an action determination model and determine an action for a received event but may also generate a virtual event or a virtual episode through the state prediction module 820.

In operation 810, the action determiner may determine an action A appropriate for the received state S, through the action determination model. The state prediction module 820 may receive S and A and may determine S′, which is a next state of S transitioned according to A, and a reward R. S, A, R, and S′ may configure the virtual event, and a series of combinations of S, A, R, and S′ may configure the virtual episode e, which may be used to train the action determination model.

The action determiner and the state prediction module 820 may perform virtual event collection and a training process repeatedly by transitioning the next state S′ to the current state S when the episode is not terminated.

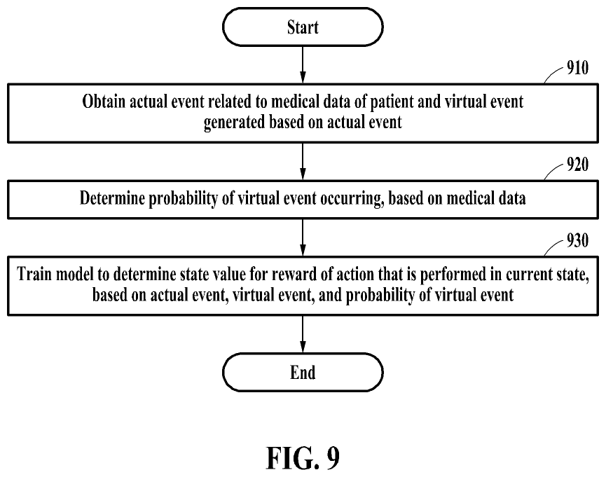

FIG. 9 is a schematic flowchart of a training operation of an electronic device for reinforcement learning, according to an embodiment.

In the following embodiments, operations may be performed sequentially but not necessarily. For example, the order of the operations may change and at least two of the operations may be performed in parallel. Operations 910 to 930 may be performed by at least one component (e.g., a processor) of an electronic device.

In operation 910, the electronic device may obtain an actual event related to medical data of a patient and a virtual event generated based on the actual event.

In operation 920, the electronic device may determine a probability of the virtual event occurring, based on the medical data. The electronic device may determine, based on a database in which the medical data is stored, the probability through a model configured to compare the virtual event to distribution of actual events in the database.

In operation 930, the electronic device may train a model to determine a state value for a reward of an action that is performed in a current state of the patient, based on the actual event, the virtual event, and the probability of the virtual event. The electronic device may determine a predicted value of the reward as the state value in the case of the actual event and may determine the state value so that the reward is predicted according to the probability of the virtual event in the case of the virtual event. The electronic device may train the model to determine the state value by using a Bellman equation, which considers the probability of the virtual event. The electronic device may further train the model, based on the state value, to select a policy for determining an action appropriate for the current state.

The virtual event may be an event in which one or more features are selected from the actual event and an event that is generated by the selected one or more features and is different from the actual event. The virtual event may further include information about a next state of the patient predicted for a medical action performed in the current state of the patient. The medical data may include information about the current state of the patient and the medical action.

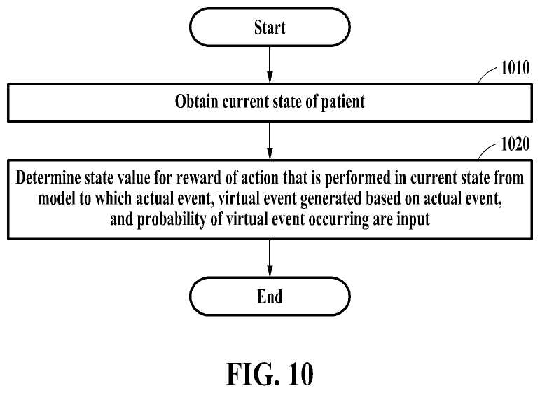

FIG. 10 is a schematic flowchart of an inference operation of an electronic device for reinforcement learning, according to an embodiment.

In the following embodiments, operations may be performed sequentially but not necessarily. For example, the order of the operations may change and at least two of the operations may be performed in parallel. Operations 1010 and 1020 may be performed by at least one component (e.g., a processor) of an electronic device.

In operation 1010, the electronic device may obtain a current state of a patient.

In operation 1020, the electronic device may determine a state value for a reward of an action that is performed in the current state of the patient from a model to which an actual event related to medical data of the patient, a virtual event generated based on the actual event, and a probability of the virtual event occurring are input.

The electronic device may determine an action appropriate for the current state, based on the state value.

The probability may be determined, based on a database in which the medical data is stored, through a model configured to compare the virtual event to distribution of actual events in the database. The virtual event may be an event in which one or more features are selected from the actual event and an event that is generated by the selected one or more features and is different from the actual event.

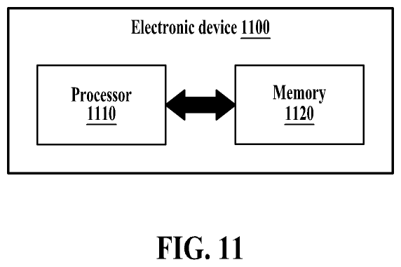

FIG. 11 is a schematic block diagram of an electronic device for reinforcement learning, according to an embodiment.

Referring to FIG. 11, an electronic device 1100 may include a processor 1110. The processor 1110 may include at least one processor. The electronic device 1100 may further include a memory 1120.

The memory 1120 may store instructions (or programs) executable by the processor 1110. For example, the instructions may include instructions for executing an operation of the processor 1110 and/or an operation of each component of the processor 1110.

The processor 1110 may be a device that executes instructions or programs or controls the electronic device 1100 and may include, for example, various processors such as a central processing unit (CPU) and a graphics processing unit (GPU). The processor 1110 may obtain an actual event related to medical data of a patient and a virtual event generated based on the actual event. The processor 1110 may determine a probability of the virtual event occurring, based on the medical data. The processor 1110 may train a model to determine a state value for a reward of a medical action that is performed in a current state of the patient, based on the actual event, the virtual event, and the probability of the virtual event.

The processor 1110 may determine a predicted value of the reward as the state value in the case of the actual event and may determine the state value so that the reward is predicted according to the probability of the virtual event in the case of the virtual event. The processor 1110 may train the model to determine the state value by using a Bellman equation, which considers the probability of the virtual event. The processor 1110 may further train the model, based on the state value, to select a policy for determining a medical action appropriate for the current state. The processor 1110 may determine, based on a database in which the medical data is stored, the probability through a model configured to compare the virtual event to distribution of actual events in the database.

The virtual event may be an event in which one or more features are selected from the actual event and an event that is generated by the selected one or more features and is different from the actual event. The virtual event may further include information about a next state of the patient predicted for an action performed in the current state of the patient. The medical data may include information about the current state of the patient and the action.

In addition, the electronic device 1100 may process the operations described above.

The components described in the embodiments may be implemented by hardware components including, for example, at least one digital signal processor (DSP), a processor, a controller, an application-specific integrated circuit (ASIC), a programmable logic element, such as a field programmable gate array (FPGA), other electronic devices, or combinations thereof. At least some of the functions or the processes described in the embodiments may be implemented by software, and the software may be recorded on a recording medium. The components, the functions, and the processes described in the embodiments may be implemented by a combination of hardware and software.

The embodiments described herein may be implemented using a hardware component, a software component, and/or a combination thereof. A processing device may be implemented using one or more general-purpose or special-purpose computers, such as, for example, a processor, a controller and an arithmetic logic unit (ALU), a DSP, a microcomputer, an FPGA, a programmable logic unit (PLU), a microprocessor, or any other device capable of responding to and executing instructions in a defined manner. The processing device may run an operating system (OS) and one or more software applications that run on the OS. The processing device also may access, store, manipulate, process, and generate data in response to execution of the software. For purpose of simplicity, the description of a processing device is singular; however, one of ordinary skill in the art will appreciate that a processing device may include a plurality of processing elements and a plurality of types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or collectively instruct or configure the processing device to operate as desired. Software and data may be stored in any type of machine, component, physical or virtual equipment, or computer storage medium or device capable of providing instructions or data to or being interpreted by the processing device. The software may also be distributed over network-coupled computer systems so that the software is stored and executed in a distributed fashion. The software and data may be stored in a non-transitory computer-readable recording medium.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of examples, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as optical discs; and hardware devices that are specifically configured to store and perform program instructions, such as read-only memory (ROM), random access memory (RAM), flash memory, and the like. Examples of program instructions include both machine code, such as one produced by a compiler, and files containing higher-level code that may be executed by the computer using an interpreter.

The above-described hardware devices may be configured to act as one or more software modules in order to perform the operations of the above-described embodiments, or vice versa.

As described above, although the embodiments have been described with reference to the limited drawings, one of ordinary skill in the art may apply various technical modifications and variations based thereon. For example, suitable results may be achieved if the described techniques are performed in a different order, and/or if components in a described system, architecture, device, or circuit are combined in a different manner, and/or replaced or supplemented by other components or their equivalents.

Therefore, other implementations, other embodiments, and equivalents to the claims are also within the scope of the following claims.

What is claimed is:

1. A method of operating an electronic device, the method comprising:
obtaining an actual event related to medical data of a patient and a virtual event generated based on the actual event;
determining a probability of the virtual event occurring, based on the medical data; and
based on the actual event, the virtual event, and the probability of the virtual event, training a model to determine a state value for a reward of an action that is performed in a current state of the patient.

2. The method of claim 1, wherein
the training of the model comprises:
in the case of the actual event, determining a predicted value of the reward as the state value; and
in the case of the virtual event, determining the state value so that the reward is predicted according to the probability of the virtual event.

3. The method of claim 1, wherein
the training of the model comprises training the model to determine the state value by using a Bellman equation, which considers the probability of the virtual event.

4. The method of claim 2, wherein
the training of the model further comprises, based on the state value, training the model to select a policy for determining an action appropriate for the current state.

5. The method of claim 1, wherein
the determining of the probability comprises, based on a database in which the medical data is stored, determining the probability through a model configured to compare the virtual event to distribution of actual events in the database.

6. The method of claim 1, wherein
the virtual event is an event in which one or more features are selected from the actual event and an event that is generated by the selected one or more features and is different from the actual event.

7. The method of claim 1, wherein
the virtual event comprises information about a next state of the patient predicted for the action performed in the current state of the patient.

8. The method of claim 1, wherein
the medical data comprises information about the current state of the patient and the action.

9. A method of operating an electronic device, the method comprising:
obtaining a current state of a patient; and
determining a state value for a reward of an action that is performed in the current state of the patient from a model to which an actual event related to medical data of the patient, a virtual event generated based on the actual event, and a probability of the virtual event occurring are input.

10. The method of claim 9, further comprising:
determining an action appropriate for the current state, based on the state value.

11. The method of claim 9, wherein
the probability is determined, based on a database in which the medical data is stored, through a model configured to compare the virtual event to distribution of actual events in the database.

12. The method of claim 9, wherein
the virtual event is an event in which one or more features are selected from the actual event and an event that is generated by the selected one or more features and is different from the actual event.

13. An electronic device comprising:
a processor configured to:
obtain an actual event related to medical data of a patient and a virtual event generated based on the actual event;
determine a probability of the virtual event occurring, based on the medical data; and
based on the actual event, the virtual event, and the probability of the virtual event, train a model to determine a state value for a reward of a medical action that is performed in a current state of the patient.

14. The electronic device of claim 13, wherein
the processor is configured to:
in the case of the actual event, determine a predicted value of the reward as the state value; and
in the case of the virtual event, determine the state value so that the reward is predicted according to the probability of the virtual event.

15. The electronic device of claim 13, wherein
the processor is configured to train the model to determine the state value by using a Bellman equation, which considers the probability of the virtual event.

16. The electronic device of claim 14, wherein
the processor is configured to, based on the state value, train the model to select a policy for determining a medical action appropriate for the current state.

17. The electronic device of claim 13, wherein
the processor is configured to, based on a database in which the medical data is stored, determine the probability through a model configured to compare the virtual event to distribution of actual events in the database.

18. The electronic device of claim 13, wherein
the virtual event is an event in which one or more features are selected from the actual event and an event that is generated by the selected one or more features and is different from the actual event.

19. The electronic device of claim 13, wherein
the virtual event comprises information about a next state of the patient predicted for the action performed in the current state of the patient.

20. The electronic device of claim 13, wherein
the medical data comprises information about the current state of the patient and the action.
